(19) United States
(12) Patent Application Publication
(10) Pub. No.: US 2008/0021526 A1
(43) Pub. Date: Jan. 24, 2008

(54) Electromagnetic Treatment of Mammals

(76) Inventor: Boris Kokorin, Kwazulu Natal (ZA)

Correspondence Address: LADAS & PARRY, 26 WEST 61ST STREET, NEW YORK, NY, 10023, US

(21) Appl. No.: 11/547,619

(22) PCT Filed: Mar. 9, 2005
(86) PCT No.: PCT/IB05/00590

§ 371 Date: Aug. 9, 2007

(30) Foreign Application Priority Data
Mar. 9, 2004 (ZA) 2004/1918

(51) Int. Cl. A61B 18/18

(52) U.S. Cl. 607/88

(57) ABSTRACT

A system for the electromagnetic treatment of mammals has a signal generator for generating an electromagnetic signal, an organic filter for supplying a desired spectrum of electromagnetic signals, and a radiating antenna for radiating the desired spectrum. In a method of treating a mammal, an electromagnetic signal is generated, the generated signal is supplied to an organic filter to provide signals with a desired spectrum and the desired spectrum of signals is radiated into a space occupied by the mammal to be treated. A feedback antenna receives the radiated signals and provides a feedback signal which is amplified and supplied to the filter. In particular, the filter may have a driven antenna, a filter element containing a biological sample and a receiving antenna in a shielded housing.

THIS INVENTION relates to the electromagnetic treatment of mammals. More particularly it relates to a system for and a method of treating mammals by means of electromagnetic radiation.

According to the invention there is provided a system for treating mammals with electromagnetic radiation, that includes a signal generator for generating an electromagnetic signal;
an organic filter for supplying a desired spectrum of signals; and
a radiating antenna for radiating the desired spectrum of signals.

Further according to the invention there is provided a method of treating mammals by means of electromagnetic radiation, that includes generating an electromagnetic signal;
supplying the signal to an organic filter to provide signals with a desired spectrum; and
radiating the desired spectrum of signals.

Both the generated electromagnetic signal and the desired spectrum of signals may be radiated.

The system may include an amplifier. The system may further have a feedback component for providing a feedback signal from the radiating antenna, which is connected to the amplifier. The amplifier may then be connected to the filter.

The filter may include a driven antenna, an organic filter element, and a receiving antenna, the filter element being located between the driven and receiving antennae. The driven antenna may then be connected to an output of the amplifier and the receiving antenna to the radiating antenna. The filter may be shielded, such that the driven antenna, the filter element, and the receiving antenna are all in a shielded container.

The signals supplied by the signal generator may be at high or very high frequencies and may be modulated at a low frequency. In a preferred embodiment the signal generator may supply a first signal at about 424 Mhz and a second signal having a range from 100 MHz to 3.5 GHz. These signals may be modulated at 1 Hz or 4 Hz.

The system and method may, in particular, be for humans and may be for treating viral or bacterial infections. The filter may then have a filter element containing a sample of the particular virus or bacteria.

The various parts of the system may use multisolenoid coils. In particular the radiating antenna may include a spheroidal multisolenoid such as that described in copending PCT application IB2005/000074 which is incorporated herein by reference.

The invention will now be described, by way of an example, with reference to the accompanying diagrammatic drawing, which shows a system for treating mammals in accordance with the invention.

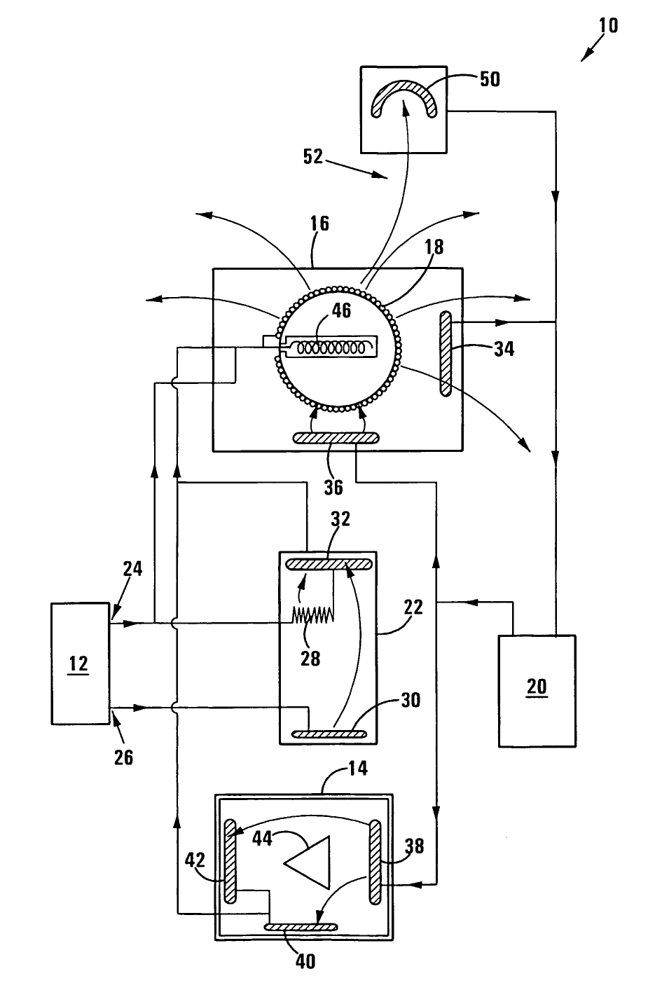

Referring to FIG. 1, a system for treating a human being for a viral infection such as HIV, is designated generally by reference numeral 10. The system 10 has a signal generator 12, a filter 14, a composite unit 16 which incorporates a radiating antenna 18, an amplifier 20, and a mixer 22.

The signal generator 12 supplies two signals-a first signal, via output 24, that is at a frequency of 424 MHz, modulated at 1 Hz; and a second signal, via output 26, that has various components at frequencies between 100 and 3500 MHz and that is modulated at a frequency of 4 Hz. These two signals are mixed in the mixer 22, by means of a simple coil 28, and two multisolenoid coils 30 and 32.

The composite unit 16 also has an auxiliary coil 46, a first feedback coil 34 and a driving coil 36. A second feedback coil 50 is positioned a suitable distance from the composite unit 16 to provide a treatment space 52 between them.

The output of the mixer 22 is supplied to the radiating antenna 18 and the auxiliary coil 46, as are the signals from the output 24 of the generator 12. Signals transmitted by the radiating antenna 18 are received by the first feedback coil 34 and the second feedback coil 50 which supply feedback signals to the amplifier 20. These signals are amplified and supplied to the driving coil 36 of the composite unit 16, and to the filter 14. The filter 14 is shielded and contains a driven antenna in the form of a coil 38 and two receiving antenna in the form of coils 40 and 42. It also has a biological, organic filter element 44. The filter element 44 is representative of the virus that is sought to be treated. Thus, appropriate signals, in accordance with the nature of the filter element 44 are supplied to the antenna 18. For example, HIV specific protein or RNA of HIV may be placed in a glass ampoule which is then placed in a wax pyramid to form the filter element. The applicant believes that the filter element 44 acts as a bandpass filter, causing signals at specific frequencies representative of and specific to, a virus or bacteria in the sample to be provided.

The radiating antenna 18 is spheroidal comprising a layer of a multisolenoid winding. The auxiliary coil 46 is also a multisolenoid coil that is located inside the radiating antenna 18. The coils 30, 32, 34, 36, 38, 40 and 42 are all spirals.

By means of the invention, the applicant believes that electromagnetic radiation may be provided which is suitable for treating viral and bacterial organisms present in a human or animal.

1. A system for treating mammals with electromagnetic radiation, that includes
a signal generator for generating an electromagnetic signal;
an organic filter for supplying a desired spectrum of signals; and
a radiating antenna for radiating the desired spectrum of signals.

2. The system as claimed in claim 1 which includes an amplifier.

3. The system as claimed in claim 2 which includes a feedback component for providing a feedback signal from the radiating antenna, the feedback component being connected to the amplifier.

4. The system as claimed in claim 3, in which the amplifier is connected to the filter.

5. The system as claimed in claim 4, in which the filter includes a driven antenna, an organic filter element and a receiving antenna, the filter element being located between the driven and receiving antennae.

6. The system as claimed in claim 5, in which the driven antenna is connected to an output of the amplifier and the receiving antenna is connected to the radiating antenna.

7. The system as claimed in claim 6, in which the driven antenna, the filter element and the receiving antenna are in a shielded container.

8. The system as claimed in claim 1, in which the signal generator supplies, in use, signals in the high to very high frequency range modulated at a low frequency.

9. The system as claimed in claim 8, in which the signal generator supplies a first signal at a frequency of about 424 MHz, and a second signal having a range from 100 MHz to 3.5 GHz

10. The system as claimed in claim 8, in which the first signal is modulated at a frequency of 1 Hz or 4 Hz.

11. The system as claimed in claim 8, in which the second signal is modulated at a frequency of 1 Hz or 4 Hz.

12. The system as claimed in claim 9, in which the first signal is supplied, in use, to the radiating antenna.

13. The system as claimed in claim 9, which includes a mixer for mixing the first and second signals.

14. The system as claimed in claim 13, in which an output of the mixer is connected to the radiating antenna.

15. The system as claimed in claim 1, in which the radiating antenna comprises a multisolenoid spheroidal antenna.

16. The system as claimed in claim 13, in which the mixer has a simple coil and two multisolenoid coils.

17. A method of treating mammals by means of electromagnetic radiation, that includes
generating an electromagnetic signal;
supplying the signal to an organic filter to provide signals with a desired spectrum; and
radiating the desired spectrum of signals.

18. The method as claimed in claim 17, which includes also radiating the generated electromagnetic signals.

19. The method as claimed in claim 17, which includes feeding back the radiated signals and amplifying them.

20. The method as claimed in claim 19, in which the amplified feedback signal is supplied to the filter.

21. The method as claimed in claim 17, which includes generating a first signal at a frequency of about 424 MHz and a second signal having a range from 100 Mhz to 3.5 GHz.

22. The method as claimed in claim 21, in which the first signal is modulated at about 1 Hz or at about 4 Hz.

23. The method as claimed in claim 21, in which the second signal is modulated at about 1 Hz or at about 4 Hz.

24. A system for treating mammals with electromagnetic radiation substantially as described in the specification with reference to the accompanying drawing.

25. A method of treating mammals by means of electromagnetic radiation substantially as described in the specification with reference to the accompanying drawing.
